(19) United States
(12) Patent Application Publication
(10) Pub. No.: US 2008/0046070 A1
(43) Pub. Date: Feb. 21, 2008

(54) CHEMICALLY TREATED EXTRACELLULAR MATRICES FOR AFFECTING THE CELLULAR RESPONSE

(76) Inventors: F. Joseph Obermiller, West Lafayette, IN (US); Michael C. Hiles, Lafayette, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/694,328

(22) Filed: Mar. 30, 2007

Related U.S. Application Data
(60) Provisional application No. 60/788,438, filed on Mar. 31, 2006.

(51) Int. Cl. A61F 2/06

(52) U.S. Cl. 623/1.24

(57) ABSTRACT

Described are preferred prosthetic valve devices including an extracellular matrix (ECM) material coated with a microtubule inhibiting agent and affixed to a stent. The stent is configured such that the coated ECM material is formed into one or more leaflets. In preferred embodiments, the prosthetic valve devices are configured for use in vascular applications.

### REFERENCE TO RELATED APPLICATIONS

The present application claims the benefit of U.S. Provisional Patent Application Ser. No. 60/788,438 filed Mar. 31, 2006, entitled “CHEMICALLY TREATED EXTRACELLULAR MATRICES FOR AFFECTING THE CELLULAR RESPONSE” which is hereby incorporated by reference in its entirety.

### TECHNICAL FIELD OF THE INVENTION

The present invention relates generally to prosthetic valve devices and, in particular, to prosthetic valve devices including a chemically treated extracellular matrix material for deployment in the vascular system.

### BACKGROUND OF THE INVENTION

A variety of extracellular matrix materials have been proposed for use in medical grafting, cell culture, and other related applications. For instance, medical grafts and cell culture materials containing submucosa derived from small intestine, stomach or urinary bladder tissues, have been proposed. See, e.g., U.S. Pat. Nos. 4,902,508, 4,956,178, 5,281,422, 5,554,389, 6,099,567 and 6,206,931. In addition, Cook Biotech Incorporated, West Lafayette, Ind., currently manufactures a variety of medical products based upon small intestinal submucosa under the trademarks SURGISIS®, STRATASIS® and OASIS®.

Medical materials derived from liver basement membrane have also been proposed, for example in U.S. Pat. No. 6,379,710. As well, ECM materials derived from amnion (see e.g. U.S. Pat. Nos. 4,361,552 and 6,576,618) and from renal capsule membrane (see WO03/002165 published Jan. 9, 2003) have been proposed for medical and/or cell culture applications.

With many medical materials, including those used in prosthetic devices, there is a potential for complications to result from their implantation. For example, when the device is employed in vascular applications, clot formation or thrombosis can result at the injured site, causing stenosis or occlusion of the blood vessel in which the device is manipulated through. Moreover, if the device is left in the vessel for an extended period of time, thrombus can form on the device, again causing stenosis or occlusion.

In an effort to combat these complications, there have been attempts to modify naturally occurring and synthetic materials in a variety of ways, including surface treatment or impregnation with any of a number of chemical or biological agents that affect the biological response to the material. For example, agents such as cytotoxins, blood thinners, steroids, non-steroid anti-inflammatory drugs (NSAIDs), and growth factors can be introduced into the material to assist in limiting stenosis, thrombosis, inflammation, and adhesions. This treated material can be tailored to a specific application in order to stimulate or down regulate appropriate cell constituents so as to obtain a more desirable cellular response to the material as compared to an untreated extracellular matrix.

By way of example, Woods et al. disclose a small intestinal submucosa (SIS) material having improved biocompatibility by virtue of it being conditioned with human umbilical vein endothelial cells (HUVECs) (Biomaterials, (25)515-525 (2004)). To produce the conditioned SIS, HUVECs were grown for 2 weeks on SIS and then removed, leaving behind an intact basement membrane. Woods et al. suggest that the above approach could be a useful step in preparing a conditioned SIS that has certain biological advantages over a native SIS.

There have also been attempts to attach modified medical materials to a prosthetic device for use in vascular applications. For example, U.S. Pat. No. 6,730,064 discloses a coated implantable prosthetic valve device, such as a coronary stent, having at least one coating layer and at least one layer of a bioactive material posited on the coating layer. In preferred embodiments, the '064 patent provides a coronary stent comprising paclitaxel posited on a non-porous coating layer affixed to a stent and suggests that such a stent may be useful in reducing restinosis.

Similarly, U.S. Pat. No. 6,624,138 discloses a method of treating tissue of a patient with a drug-loaded biological material affixed onto a prosthetic valve device. The '138 patent discloses a wide range of drugs suitable for use in the device, with preferred drugs including paclitaxel, among others.

Despite work in these areas, there remain needs for alternative and improved medical materials, as well as methods and devices related to these materials. The present invention addresses these needs.

### SUMMARY OF THE INVENTION

In one aspect, the present invention provides a prosthetic valve device for deployment in the vascular system. The prosthetic valve device comprises an extracellular matrix (ECM) material coated with a microtubule inhibiting agent and affixed to a stent. The stent is configured such that the coated ECM material is formed into one or more leaflets. The microtubule inhibiting agent can be incorporated into the ECM material in an effective amount to reduce the extent of retraction of the ECM material upon remodeling.

The present invention further provides a method for preparing a prosthetic valve device for deployment in the vascular system. The method comprises providing a stent and a sheet of extracellular matrix (ECM) material. The ECM material is coated, at least in part, with a microtubule inhibiting agent and is affixed to the stent. The stent is configured such that the coated ECM material is formed into one or more leaflets.

Further provided by the invention is a method for treating a patient. The method comprises providing a prosthetic valve device of the invention and implanting the valve device into a bodily passage of the patient.

Additional embodiments of the invention relate to uses of such treated ECM materials as described above for treating patient tissue in locations other than those where valves are desired. In certain embodiments, the ECM materials are used in ophthalmic applications, such as for grafting in the treated eye.

Additional embodiments as well as features and advantages of the invention will be apparent from the descriptions herein.

### BRIEF DESCRIPTION OF THE DRAWINGS

FIG. 1 provides a side view of a prosthetic valve device of the invention.

FIG. 2 provides a left side view of the prosthetic valve device depicted in FIG. 1.

FIG. 3 provides a right side view of the prosthetic valve device depicted in FIG. 1.

### DETAILED DESCRIPTION

The present invention provides a prosthetic valve device for deployment in the vascular system. The prosthetic valve device comprises an extracellular matrix (ECM) material coated with a microtubule inhibiting agent and affixed to a stent, wherein said stent is configured such that the coated ECM material is formed into one or more leaflets.

For the purposes of promoting an understanding of the principles of the invention, reference will now be made to the embodiment illustrated in the drawings and specific language will be used to describe the same. It will nevertheless be understood that no limitation of the scope of the invention is thereby intended. Any alterations and modifications in the illustrated device and method, and further applications of the principles of the invention as illustrated therein are herein contemplated as would normally occur to one skilled in the art to which the invention relates are included.

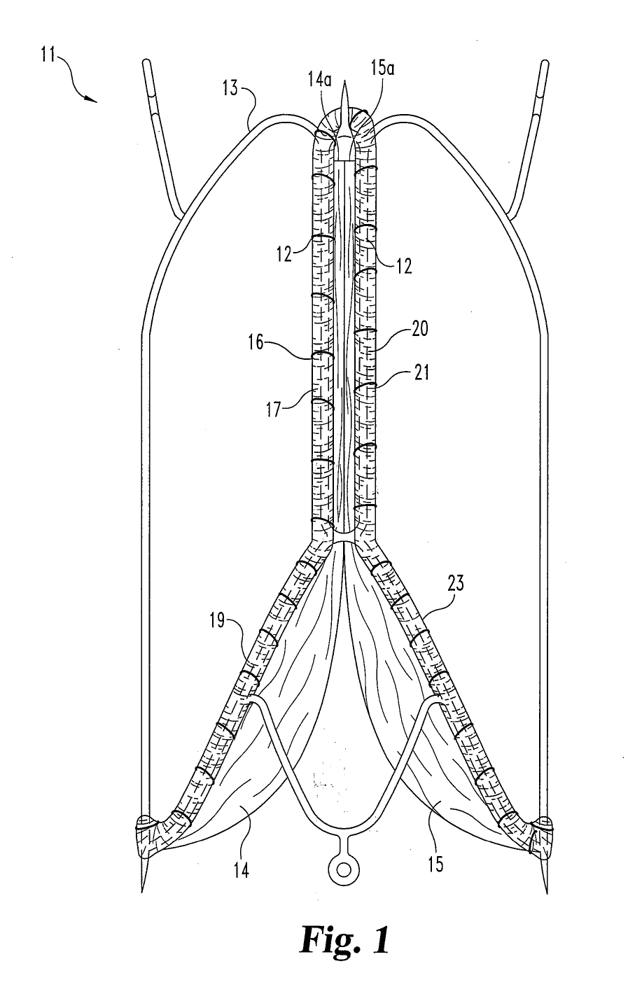

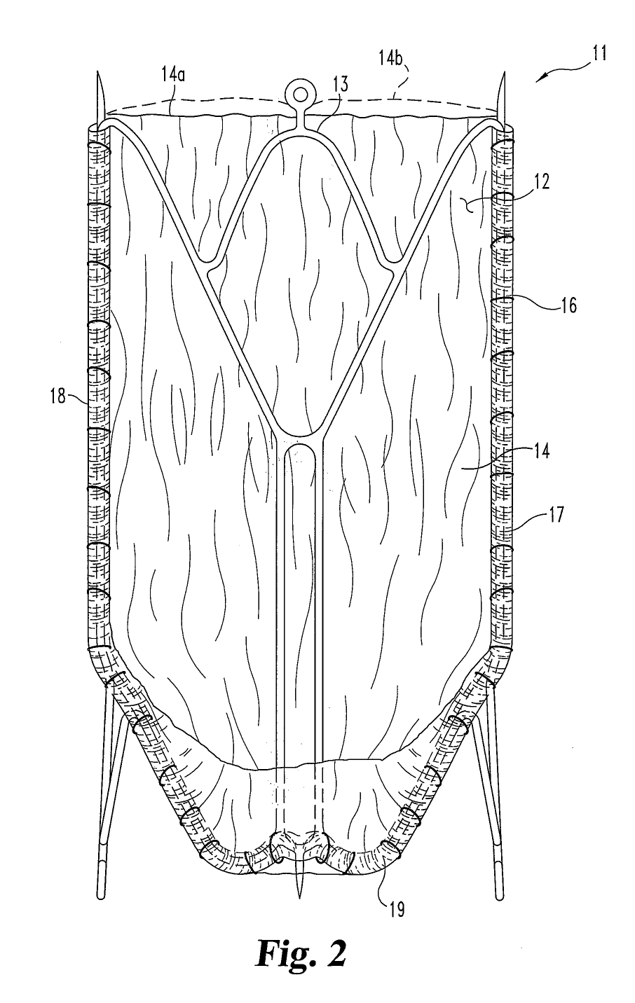

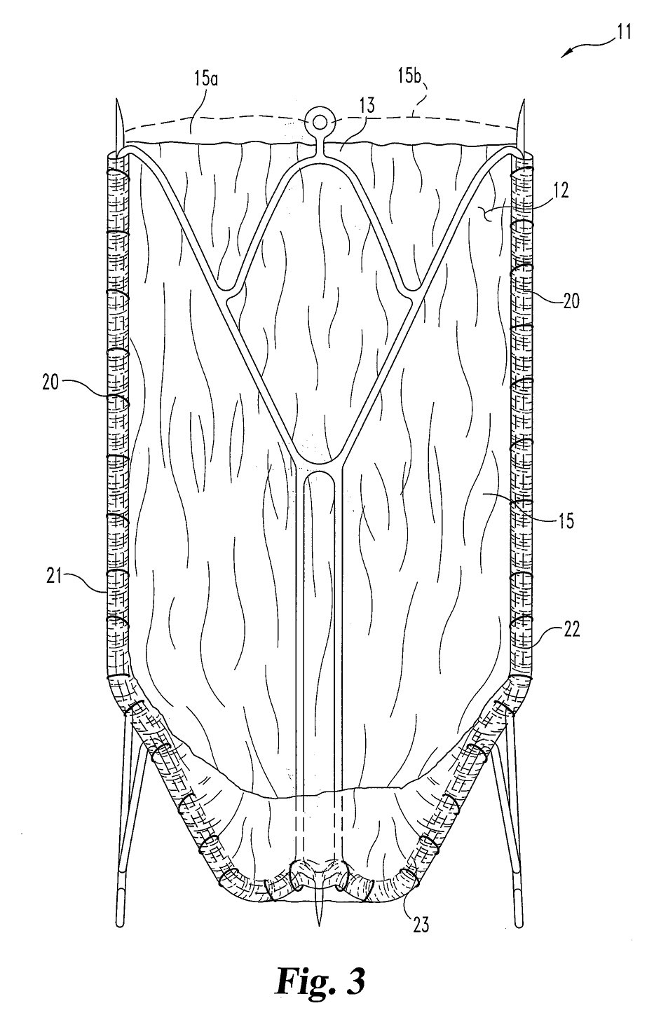

With reference now to FIGS. 1-3, depicted are various side views of a prosthetic valve device 11 of the invention. The extracellular matrix material 12 is coated with a microtubule inhibiting agent and is attached to a frame element 13 to provide two leaflets 14 and 15 in an original configuration for implantation in a patient. In particular, FIG. 1 provides a side view of prosthetic valve device 11 taken in a direction parallel to the coapting upper edges 14a and 15a of leaflets 14 and 15. FIG. 2 provides a view of the device 11 depicted in FIG. 1 taken from the left side. FIG. 3 provides a view of the device 11 depicted in FIG. 1 taken from the right side. Device 11 is particularly well suited for vascular applications, such as implantation into a vascular passage of a patient.

As can be seen from FIGS. 1-3, leaflets 14 and 15 include respective free edges 14a and 15a for coaptation with one another and respective fixed edges 16 and 20 that will each be forced against the wall of a vascular vessel upon implantation of device 11 in a path that partially circumscribes the vessel wall so as to each form a blood-capturing element. In the device 11 illustrated, the path of leaflet edge contact with the vessel wall includes substantial portions that extend essentially longitudinal along the vessel wall that connect to a cup-forming portion that extends both longitudinally along the vessel wall and circumferentially around the vessel wall. In particular, the fixed edge 16 of leaflet 14 includes opposite longitudinally-extending portions 17 and 18 each extending to an opposite side of a cup-forming portion 19. Correspondingly, the fixed edge 20 of leaflet 15 includes opposite longitudinally-extending portions 21 and 22 each extending to an opposite side of cup-forming portion 23.

Prosthetic valve device 11 includes an excess of the ECM material 12 carried upon the frame element 13. The excess ECM material may be provided in a direction transverse to (e.g. perpendicular to) and/or in a direction parallel to a longitudinal axis of the frame 13. This renders the leaflets 14 and 15 more slack than that which occurs in the target remodeled configuration in which the leaflets comprise tissue of the patient into which the valve device 11 is implanted. Thus, in the original or implanted configuration shown in FIGS. 1-3, the leaflets 14 and 15 may be generally more billowed in the open condition of the valve than in the target, remodeled configuration of the valve 11. In other ways to consider the excess ECM material 12, the leaflets 14 and 15 may each present a greater leaflet surface area in the implanted configuration than in the target configuration, and/or from a cross-sectional standpoint, the leaflets 14 and 15 may define at least one cross-sectional path that is longer in the implanted configuration than in the target configuration.

The amount of contacting or coapting leaflet area can be expressed in a number of different ways. The length of coaptation (e.g., LOC) in the original configuration for implant is desirably at least about 2 mm and may be as much as about 50 mm or more depending on the configuration of the valve prosthesis. In certain embodiments of the invention, the length of coaptation can be within the range of about 5 to about 30 mm, more typically about 5 to about 15 mm, in the original configuration for implant. The length of coaptation can represent a substantial percentage of the overall length of the valve prosthesis, for example, at least about 5%, or at least about 10%, of the overall length of the prosthesis. In certain embodiments, the length of coaptation of the leaflets represents 10% to 80% of the length of the overall device, typically about 30% to about 60%, and more typically about 35% to about 55%.

In additional aspects of the invention, a long length of coaptation can be provided by orienting the outer leaflet edges substantially longitudinally along the frame in close proximity to one another over a significant distance. Thus, with reference to FIGS. 1-3 for purposes of illustration, outer leaflet edge portion 17 of leaflet 14 is configured to contact along the vessel wall in close proximity to outer leaflet edge portion 21 of leaflet 15 over a significant distance, for example 2 to 50 mm, typically about 5 to about 30 mm, and more typically about 5 to about 15 mm. The same would be true for the leaflet edge portions tracking along the opposite side of the vessel wall (e.g., edge portions 18 and 22, FIGS. 1-3). It is preferred that the leaflet edges remain in close proximity over these distances, for example within about 5 mm, more preferably within about 3 mm, and most preferably within about 1 mm. It will be understood that this close proximity may involve having leaflet edges track closely with one another along the vessel wall, or may have them being attached along essentially the same path (e.g., both along a single strut of a frame) and thus exhibiting essentially no separation from one another as they pass along the vessel wall.

It is advantageous to use a remodelable material for the prosthetic valve devices and methods of the present invention, and particular advantage can be provided by including a remodelable collagenous material. Such remodelable collagenous materials can be provided, for example, by collagenous materials isolated from a suitable tissue source from a warm-blooded vertebrate, and especially a mammal. Such isolated collagenous materials can be processed so as to have remodelable properties and promote cellular invasion and tissue infiltration. Remodelable materials may be used in this context to promote cellular growth within the site in which a medical material of the invention is implanted.

Reconstituted or naturally-derived collagenous materials can be used in the present invention. Such materials that are at least bioresorbable will provide advantage in the present invention, with materials that are bioremodelable and promote cellular invasion and ingrowth providing particular advantage.

Suitable bioremodelable materials can be provided by collagenous extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

In addition to the above, the ECM material as used in the prosthetic valve devices of the invention is coated, at least in part, with a microtubule inhibiting agent. By “coated” is meant that the microtubule inhibiting agent is applied to an ECM material so as to cover a designated portion of a surface of the ECM material. A sufficient coating is achieved when the microtubule inhibiting agent effectively reduces the degree of cellular invasion where it is applied. For example, the microtubule inhibiting agent is applied to selective portions of a surface of the ECM material to form a coating over these selective portions. In preferred embodiments, the microtubule inhibiting agent is applied to a substantial portion of a surface of the ECM material to form a coating which substantially covers the entire surface area of the surface to which it is applied. In addition to reducing the degree of cellular invasion, the addition of an effective amount of a microtubule inhibiting agent reduces the extent of retraction of the ECM material upon remodeling.

Any suitable microtubule inhibiting agent may be used in the context of the present invention. Suitable microtubule inhibiting agents include, for example, 2-methoxyestradiol, an epothilone, colchicine, dolastatin 15, nocodazole, paclitaxel, podophyllotoxin, rhizoxin, vinblastine, vincristine, vindesine, and vinorelbine (navelbine). Preferably, the microtubule inhibiting agent comprises Taxol® or a derivative thereof. In more preferred embodiments, the microtubule inhibiting agent is a derivative of Taxol®, such as paclitaxel.

The microtubule inhibiting agent can be applied to an ECM material by any suitable means. Suitable means include, for example, spraying, impregnating, dipping, etc. The microtubule inhibiting agent can be applied to the ECM material either before or after the ECM material is affixed to a stent. Similarly, if other chemical or biological agents are included in the ECM material, the microtubule inhibiting agent can be applied either before, in conjunction with, or after these other agents.

The amount of microtubule inhibiting agent included in the ECM material of the invention may vary depending on its desired use. Typically, the area of a surface of the ECM comprises from about 0.01 mg to about 10 mg of the microtubule inhibiting agent per cm². More preferably, the ECM comprises from about 0.1 mg to about 5 mg of a microtubule inhibiting agent per cm². The microtubule inhibiting agent can be incorporated into the ECM material in an amount that is effective to decrease the extent of shrinkage or contraction of the ECM material when it has been implanted in the vascular system and subsequently remodeled. For instance, the agent can be incorporated throughout the thickness of the ECM leaflets, or on a surface or face thereof, or discrete portions thereof, or any other suitable configuration. In certain embodiments, at least the portion of the ECM or the implanted device that serves as the valve leaflet(s) will be coated with and/or impregnated with the agent. For example, it may be desirable to coat the surface(s) of the valve leaflets that face the vascular wall. In other embodiments, the surface(s) that face the vascular wall will be uncoated and the surface(s) that face the inner lumen of the vascular passage will be coated with the agent. In still other embodiments, each surface of a valve leaflet i.e., both the surface(s) facing the vascular wall and the inner lumen, will be coated with the agent.

The prosthetic valve devices of the invention are implanted into a bodily passage attached to a frame, such as a self-expanding or otherwise expandable stent, both to form biocompatible coverings such as sleeves and to form leaflets or other valve structures (see, e.g. WO 99/62431 published Dec. 9, 1999 and WO 01/19285 published Mar. 22, 2001). In one mode of forming a valve structure, the ECM material can be attached to a stent in a fashion whereby it forms one, two, or more leaflets, cusps, pockets or similar structures that resist flow in one direction relative to another. In a specific application, such devices constructed as vascular valves are implanted to treat venous insufficiencies in humans, for example, occurring in the legs.

Illustratively, the expandable member(s) incorporated into prosthetic valve devices of the invention may be any one of wide variety of stent devices that have been or are currently commercially available. Stent devices provide a supporting framework structure that may take many forms. Open or perforate stents are known, which may include a network of struts or wire-like elements. The stent device used in the present invention may be of any suitable design including, for example, both forcibly expandable and self-expanding stents. As is known, forcibly expandable stents can be provided and delivered in a contracted state, and then expanded upon the application of a force, e.g. an outward radial force, to the stent. Commonly, the outward radial force is provided by an expandable member, such as a balloon, received within the contracted stent structure. Several such “balloon-expandable” stents are currently available on the commercial market. Self-expanding stents can be designed so as to be configurable to and held in a contracted state for delivery, and then released at a target site, whereupon they expand on their own. Stents are also known that take on a contracted state, but expand in response to a conditional change, e.g. a change in temperature such as may be incurred in a temperature transition from a first temperature below the body temperature of a patient, to the body temperature of the patient. Stents having these or other characteristics may be used in embodiments of the present invention.

Stents or other expandable support members may be made from metallic or non-metallic material, or both. The non-metallic material can suitably be a synthetic polymeric material, including for example bioresorbable and/or non-bioresorbable plastics. Materials commonly used in stent construction include biologically compatible metals, e.g. stainless steel, titanium, tantalum, gold, platinum, copper and the like, as well as alloys of these metals; synthetic polymeric materials; low shape memory plastic; a shape-memory plastic or alloy, such as nitinol; and the like.

Just to identify a few non-limiting examples, suitable stents for use in the invention include the Zilver stent, Gianturco-Roubin stent, the Palmaz-Schatz stent, Wallstent, Mammotherm stent, Symphony stent, Smart stent, Perflex, AVE, Intrastent, and Herculink stents, self-expanding Instent, Gianturco Z-stent, Ultraflex nitinol mesh stent, Esophacoil stent, Gianturco Z tracheobronchial tree stent, and the Wallstent tracheobronchial endoprosthesis.

Certain embodiments of the invention provide prosthetic valve devices including an expandable member, e.g. as described above, associated with a full or partial covering of an extracellular matrix material on an inner and/or outer surface of the expandable member. In some embodiments, the ECM material is associated in a unique manner with the expandable member. For example, the ECM material may be contoured snugly around or completely embed elements of the expandable member to assist in maintaining the attachment of the ECM material to the expandable member. This may avoid, reduce, or simplify the need for other mechanical attachments, such as sutures, to hold the ECM material to the expandable member. It may also in some forms provide a unique, relatively fixed association of the ECM material with the expandable member or elements thereof, even during contraction and/or expansion of the expandable member.

In one embodiment of the invention, the ECM material is attached to the stent or other expandable member by pressing or otherwise forcing the ECM material against surfaces of the expandable member while the ECM material is in a relatively conformable state, and then converting the ECM material to a less conformable state. In this manner, the ECM material while conformable can locally contour to elements of the expandable member, e.g. struts or other wire-like elements of a stent, and when converted to its relatively less conformable state will maintain that contour to the elements of the expandable member. As a result, the attachment of the ECM material to the expandable member will be facilitated. Further, the ECM material may have at least some shape memory properties such that if converted back to a conformable state, a contoured relation between the elements of the expandable member and the ECM material will still exist.

In preferred aspects of the invention, the ECM material will be hydratable, and will be relatively more conformable when hydrated than when dried. In this fashion, the ECM material while in a hydrated state can be forced against an inner and/or outer surface of the expandable member sufficiently to locally contour the ECM material to elements of the expandable member, and then dried while maintaining that force to achieve an attachment of the ECM material to the expandable member. Advantageously, a vacuum pressing operation can be utilized to both force the ECM material against the expandable member and to dry the ECM material.

In other embodiments of the invention, ECM material positioned upon one side of the expandable member (e.g. inside or outside the lumen of a stent) can be attached through open areas of the expandable member to a material on the other side of the expandable stent, so as to facilitate attachment of the ECM material to the expandable element. In some inventive forms, the attachment of the two opposing materials can be over essentially all contacting areas of the two materials, so as to effectively fix the relation between entrapped elements of the expandable member and the covering, so that no substantial sliding of the elements within the surrounding covering is observed. In still other forms, the two opposed materials can be attached to one another in a manner including fusion of the two materials to one another, so that elements of the expandable member are effectively embedded within a mass of inner and outer ECM materials. When the inner and outer materials are the same, then the expandable member elements become embedded in a mass of the same material.

Again, in these embodiments of the invention, the ECM materials used may have a relatively conformable state during a time in which they are forced against the expandable member elements and against themselves, and then be attached and converted to a less conformable state. The ECM materials can thereby locally contour to elements of the expandable member while in a conformable state, and upon attachment to each other will effectively and closely embed the expandable member elements. Again, in these aspects of the invention, the preferred ECM materials will be hydratable, and will be relatively more conformable when hydrated than when dried. The ECM material while hydrated state can thus be forced against an inner and/or outer surface of the expandable member, and attached and dried. Advantageously, a vacuum pressing operation can be utilized to both force the ECM material against the expandable member and to dry the ECM material. Also advantageously, the ECM material(s) is/are desirably of a character so as to form an attachment to one another by virtue of being dried while compressed against each other. Dehydration of the ECM materials in forced contact with one another effectively bonds the materials to one another, even in the absence of other agents for achieving a bond, although such agents can be used while also taking advantage at least in part on the dehydration-induced bonding. With sufficient compression and dehydration, the two ECM materials can be caused to form a generally unitary collagenous structure embedding the expandable member elements. Vacuum pressing operations, and the closely embedded nature that they can characteristically impart to the ECM material(s) and expandable member element(s), are highly advantageous and preferred in these aspects of the invention.

In this regard, suitable equipment for use for vacuum pressing in the present invention can be commercially obtained. One such vacuum pressing apparatus is commercially available from Zip-Vac East, Incorporated, Kennesaw, Ga. This vacuum pressing apparatus has a flexible chamber that has a vacuum drawn on it, which pulls the flexible boundaries of the chamber onto and around the article in the chamber. The vacuum also removes water from hydrated materials within the chamber.

With respect to the above, the invention provides a method for preparing a prosthetic valve device for deployment in the vascular system. The method comprises providing a stent and a sheet of ECM material. At least a portion of the ECM material is coated with a microtubule inhibiting agent and is affixed to the stent. The stent is configured such that the coated ECM material is formed into one or more leaflets.

In preferred embodiments, the coated ECM material of the invention will have a post-remodeling thickness profile of from about 50 to about 5000 microns, more preferably about 100 to about 500 microns. ECM materials having a thin profile after remodeling are expected to be advantageous for use in vascular applications such as valve leaflet applications.

Devices of the invention are desirably adapted for deployment within the vascular system, and in particularly preferred embodiments, devices of the invention are adapted for deployment within the venous system. In this respect, the invention provides a method of treating a patient. The method comprises providing a prosthetic valve device of the invention, inserting the device into a vascular passage of the patient, and delivering the device to a desired location in the vascular passage. Preferably, the device is used to treat a patient for venous insufficiency. Accordingly, preferred devices, such as device 11, are adapted as venous valves, for example for percutaneous implantation within veins of the legs or feet, to treat venous insufficiency.

The prosthetic valve devices of the invention can be attached to a wall of a bodily passage in any suitable manner. Typically, the manner in which the valve device is attached to a bodily passage will depend on the type of valve device employed. Preferably, the prosthetic valve device is attached to a wall of a vein or other vascular vessel surgically. Such a surgical procedure typically comprises suturing or otherwise physically connecting the edges of the prosthetic valve device to the luminal surface of a vein or other vascular vessel. Other potential surgical attachment procedures include, for example, stapling, bonding or otherwise adhering the edges of the prosthetic valve device to the luminal surface of a vein or other vascular vessel.

In certain embodiments, a prosthetic valve device of the invention may comprise adaptations which assist in attachment to the vessel wall. Vascular valves suitable for this use include those described in, for example, International PCT Patent Application No. WO 04/289253. Briefly, the '253 PCT application describes a bicuspid valve including a lip or other reinforcement means along the edges of the leaflets. The lip incorporates adaptations for attachment to the vessel wall. For example, the lip can include barbs or hooks, or can alternatively or additionally, can be provided with a biocompatible adhesive sufficient to secure the lip to the vessel wall. Tissue welding techniques as known in the art are also contemplated.

Where the inventive prosthesis is to be used to provide a venous valve, the prosthesis can be implanted above, below, or at the location of a native venous valve in the patient. Moreover, a plurality of the prosthetic valve devices can be implanted in a given vein to treat venous insufficiency or other similar disorders.

In certain other embodiments, ECM material treated with microtubule inhibiting agents as described herein can be formed for use in applications other than vascular valves, including for instance ophthalmic applications. For example, at least a portion of the collagenous ECM material can be coated or otherwise provided with a microtubule inhibiting agent and implanted into a mammal to repair or replace damaged or diseased portions of the eye, e.g., a cornea or other ocular surface. Illustratively, agent-treated ECM sheet grafts as described herein can be used for reconstructing the conjunctival surface of the eye, e.g., following the excision of neoplasia. Agent-treated ECM sheet grafts as described herein can also be used as tectonic supports, epithelialization substrates or superficial patch grafts in the treatment of ocular conditions. Ocular surface reconstructions can be performed with the ECM graft alone or in combination with epithelial cells from the limbus, e.g., obtained from the patient, an allograft donor, or a cell line. The cells can, for example, be seeded on the ECM graft without culturing, e.g., immediately prior to implantation, or can be cultured on the ECM graft prior to implantation. In other embodiments, the collagenous ECM material can be formed into a fluidized composition and mixed with a microtubule inhibiting agent. The resulting fluidized composition can be injected to a desired location in the eye to repair or replace damaged or diseased portions thereof. In these respects, the invention provides a method for repairing or replacing a damaged or diseased ophthalmic structure in a mammal. The method comprises (a) providing a collagenous ECM material including a microtubule inhibiting agent, and (b) delivering the collagenous ECM material including the microtubule inhibiting agent to the eye. In certain embodiments, at least a portion of the damaged or diseased eye structure can be removed from the eye of the mammal (including a human patient) prior to delivering the collagenous ECM material. If needed, the collagenous ECM material can be further secured to the eye such as, for example, by the use of sutures, staples, adhesives, and the like, as generally known in the art.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. In addition, all publications cited herein are indicative of the abilities of those of ordinary skill in the art and are hereby incorporated by reference in their entirety as if individually incorporated by reference and fully set forth.

What is claimed is:

1. A prosthetic valve device for deployment in the vascular system, comprising:
an extracellular matrix (ECM) material coated with a microtubule inhibiting agent and affixed to a stent, wherein said stent is configured such that the coated ECM material is formed into one or more leaflets.

2. The prosthetic valve device of claim 1, wherein said microtubule inhibiting agent comprises paclitaxel or a derivative thereof.

3. The prosthetic valve device of claim 2, wherein said microtubule inhibiting agent comprises paclitaxel.

4. The prosthetic valve device of claim 1, wherein said ECM material comprises submucosa.

5. The prosthetic valve device of claim 4, wherein said submucosa is intestinal, urinary bladder or stomach submucosa.

6. The prosthetic valve device of claim 5, wherein said submucosa is small intestinal submucosa (SIS).

7. The prosthetic valve device of claim 1, wherein said coated ECM material is formed into more than one leaflet.

8. The prosthetic valve device of claim 1, wherein said device is configured to form a vascular valve.

9. The prosthetic valve device of claim 8, wherein said vascular valve is a unidirectional valve.

10. The prosthetic valve device of claim 1, wherein said ECM material has a thickness profile of from about 0.1-10 mm after remodeling.

11. A method for preparing a prosthetic valve device for deployment in the vascular system, comprising:
providing a stent and a sheet of extracellular matrix (ECM) material, coating at least a portion of the ECM material with a microtubule inhibiting agent, affixing the coated ECM material to the stent, and configuring the stent such that the coated ECM is formed into one or more leaflets.

12. The prosthetic valve device of claim 11, wherein said microtubule inhibiting agent comprises paclitaxel or a derivative thereof.

13. The prosthetic valve device of claim 12, wherein said microtubule inhibiting agent comprises paclitaxel.

14. The method of claim 11, wherein said ECM material comprises submucosa.

15. The method of claim 14, wherein said submucosa is intestinal, urinary bladder or stomach submucosa.

16. The method of claim 15, wherein said submucosa is small intestinal submucosa.

17. The method of claim 11, wherein said microtubule inhibiting agent is topically applied to the ECM material.

18. The method of claim 17, wherein said microtubule inhibiting agent is sprayed onto the ECM material.

19. The method of claim 11, wherein said coated ECM material is formed into more than one leaflet.

20. The method of claim 11, wherein said device is configured to form a vascular valve.

21. The method of claim 20, wherein said vascular valve is a unidirectional valve.

22. The method of claim 20, wherein said ECM material has a thickness profile of from about 50 to about 500 microns.

23. A method for treating a patient, comprising:
providing the prosthetic valve device of claim 1, inserting the prosthetic valve device into a vascular passage of the patient, and delivering said device to a desired location in the vascular passage.

24. The method of claim 23, wherein the patient is treated for venous insufficiency.
